(19) United States
(12) Patent Application Publication
(10) Pub. No.: US 2022/0016067 A1
(43) Pub. Date: Jan. 20, 2022

(54) NOVEL ANTI-INFLAMMATORY COMPOUND, PRODUCING METHOD AND USE THEREOF

(71) Applicant: CHALLENGE BIOPRODUCTS CO., LTD., Yun-Lin Hsien (TW)

(72) Inventors: Ching-Kuan Lin, Yun-Lin Hsien (TW); I-Fan Lin, Yun-Lin Hsien (TW); Ping-Chung Kuo, Yun-Lin Hsien (TW); Ping-Hong Chen, Yun-Lin Hsien (TW); Tze-Cheng Tzen, Yun-Lin Hsien (TW)

(21) Appl. No.: 17/023,738

(22) Filed: Sep. 17, 2020

(30) Foreign Application Priority Data
Jul. 16, 2020 (TW) 109124130

(51) Int. Cl. A61K 31/225

(52) U.S. Cl. CPC A61K 31/225

(57) ABSTRACT

A novel anti-inflammatory compound has the general formula (I):

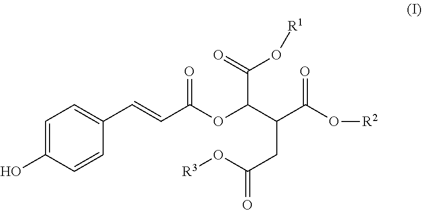

wherein the R¹, R² and R³ are same or different, and independently selected from a group consisting of H, halo, alkyl, alkenyl, alkynyl, cyclyl, heterocyclyl, alkoxyl, aryl, heteroaryl, alkylaryl and CF₃. An anti-inflammatory composition includes the compound of general formula (I) or the salt, ester and/or hydrate thereof. The anti-inflammatory compound may be separated from a fruit extract, such as pineapple extract, and exhibits inhibitory effects on stimulated inflammatory response.

### FIELD OF THE INVENTION

The present invention relates to an anti-inflammatory compound separated from a fruit extract. In particular, the present invention relates to a PGE2 analog separated from pineapple extract.

### BACKGROUND OF THE INVENTION

Cyclooxygenase (COX) is an enzyme involved in the synthesis of prostaglandin family. Since 1990, it has been found that many cells contain two kinds of COX, named COX-1 and COX-2 respectively. COX-1 maintains the integrity of gastrointestinal capillaries and has a protective effect on the gastric mucosa. It may synthesize Thromboxane A2 to regulate platelet aggregation, and achieve the function of controlling the blood flow of the kidney by regulating renal vascular resistance, blood flow, sodium ion excretion and ADH (antidiuretic hormone) antagonism.

COX-2 is an enzyme induced by inflammation, and is almost undetectable in normal cells. It is only induced under a diseased state, such as induced by an inflammatory stimulation or cytokines, and converts arachidonic acid into PGE2 (Prostaglandin E₂), PGF2α (Prostaglandin F2α), thromboxane and other prostaglandins. PGE2 is a proinflammatory factor, which acts on various tissues through autocrine and paracrine. It is known that PGE2 is one of the most important molecules in the inflammation response. Therefore, studies have suggested that suppression of the formation or functions of PGE2 may alleviate the symptoms of inflammation (i.e., redness, swelling, heat and pain).

Macrophages will be induced to produce an immune response and inflammation when activated by bacteria; it further makes the intracellular nitric oxide synthase (iNOS) to releases nitric oxide (NO). Although an appropriate level of nitric oxide in the human body has the effect of destroying foreign bacteria, over-production of NO will cause acute or chronic inflammation of the tissues. It is reported that through activating its receptor protein EP2 (Prostaglandin E₂ receptor 2), PGE2 improves the expression of iNOS and the production of NO by regulating the cAMP/PKA/Ca²⁺ signaling pathway (Tzeng S F et al., Glia. 15; 55(2): 214-223, 2007).

Many fruits and vegetables are known to have anti-inflammatory effects, including kiwi, pineapple, green papaya, banana, grape, cranberry, tomato and broccoli. In addition, curcumin exhibits anti-oxidant functions to help the liver detoxify and reduce inflammation. Green tea can reduce cell injury and inflammation because it has a high content of catechins. It is generally believed that pineapple contains a unique pineapple enzyme (commonly known as bromelain), which can effectively relieve pain and inflammation by stimulating the production of cytoplasmic elements to prevent inflammation.

Actually, bromelain is not a single substance, but a combination of various protein digestive enzymes found in the juice and stem of pineapple plant. bromelain is active in both the acidic environment of the stomach and the alkaline environment of the small intestine, making it effective in helping digestion and suitable for alleviating indigestion.

In addition to helping digestion, bromelain is also commonly used in the recoveries from sports injuries or surgery, and to relieve symptoms of sinusitis and phlebitis. However, current studies on the effects of bromelain in postoperative inflammation and swelling are not consistent.

The commercially available bromelain is produced by crushing, squeezing of pineapple stems, then centrifugation, ultrafiltration and freeze drying to form a yellowish powder, which is usually sold in a form of powder packets, tablets or capsules. The commercially available bromelain is essentially a mixed substance with an unclear composition.

As bromelain is a combination of pineapple enzymes, a kind of protein, it can be easily destroyed and lose activity during heating. Therefore, the present invention first attempts to separate and purify a single active compound with anti-inflammatory effect from the water extract of pineapple. Through molecular identification, it is found that the compound contains coumaroyl and an isocitrate portion. Experiments have shown that the anti-inflammatory compound can effectively inhibit the production of nitric oxide and the expression of iNOS and NFκB (nuclear factor kappa B) proteins induced by LPS (Lipopolysaccharide) in macrophages. Furthermore, the anti-inflammatory compound is similar in the molecular structure to PGE2, and also has the ability of binding to the PGE2 receptor EP4 (prostaglandin E₂ receptor 4).

### SUMMARY OF THE INVENTION

Accordingly, in one aspect, the present invention provides an anti-inflammatory compound of general formula (I), or its pharmaceutically acceptable salts, esters, or hydrates:

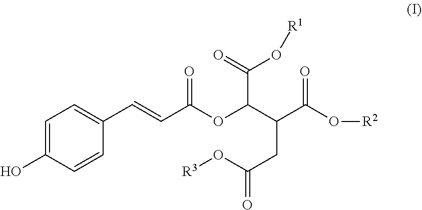

wherein the R¹, R² and R³ are same or different, and independently selected from a group consisting of H, halo, alkyl, alkenyl, alkynyl, cyclyl, heterocyclyl, alkoxyl, aryl, heteroaryl, alkylaryl and CF₃.

In certain embodiments of the invention, at least one of the R¹, R² and R³ is H. In a preferable embodiment of the invention, all of the R¹, R² and R³ are H. In other embodiments of the invention, at least one of the R¹, R² and R³ is substituted or unsubstituted C₁₋₁₀ alkyl. In a preferable embodiment of the invention, all of the R¹, R² and R³ are substituted or unsubstituted C₁₋₁₀ alkyl.

Preferably, the substituted or unsubstituted C₁₋₁₀ alkyl is a substituted or unsubstituted C₁₋₆ alkyl, and more preferably is a substituted or unsubstituted methyl, ethyl, propyl, isopropyl or butyl.

Preferably, the alkoxyl is a substituted or unsubstituted C₁₋₆ alkoxyl, and more preferably is a substituted or unsubstituted methoxy, ethoxy, propoxy, isopropoxy, butoxy, pentoxy or hexoxy. Preferably, the alkenyl is a C₂₋₆ alkenyl, more preferably a substituted or unsubstituted vinyl, allyl, butenyl or pentenyl.

The compound of present invention also comprises pharmaceutically acceptable salts, esters, hydrates and other derivative forms thereof. The said pharmaceutically acceptable salts comprise pharmaceutically acceptable alkaline salts, including alkali metal salts (such as sodium salt, potassium salt), alkaline earth metal salts (such as calcium salt, magnesium salt), ammonium salt, and the salts formed with an organic base (e.g. dicyclohexylamine and N-methyl-D-glucosamine).

In another aspect, the present invention provides a method for preparing the compound of general formula (I), comprising the steps of: obtaining a water extract of a fruit, using dichloromethane and ethyl acetate for partition extraction, and then applying the organic layer obtained to Sephadex LH-20 column for purifying and separating to obtain the compound of general formula (I) with anti-inflammatory activity.

In another aspect, the present invention provides an anti-inflammatory composition, comprising the compound of general formula (I), or a pharmaceutically acceptable salt, ester or hydrate thereof, and a pharmaceutically acceptable carrier, excipient or diluent.

### BRIEF DESCRIPTION OF THE DRAWINGS

FIG. 1 is a flow chart of the method of the present invention for separating and purifying the compound of general formula (I) from pineapple extract as described in Example 1.

FIG. 2 shows a 320 nm spectrum of the pineapple extract obtained by the LC-MS/MS (Liquid chromatography—mass spectrometry/mass spectrometry) analysis.

FIG. 3A shows the 320 nm spectrum and FIG. 3B shows the UV-Vis spectrum of Pineapplin PL6, a compound of general formula (I).

FIG. 4 shows the identified molecular structure of Pineapplin PL6, with the molecular formula of C₁₅H₁₄O₉ and molecular weight of 338.27.

FIG. 5A shows the effect of Pineapplin PL6 and FIG. 5B shows the effect of pineapple extract on cell viability in RAW264.7 cells. The data are expressed as mean±SD for three independent experiments. In the figure, “***” above the bar means p<0.001, indicating statistically significant differences from treatments with control group.

FIG. 6 shows the effect of Pineapplin PL6 on reducing LPS-induced NO production in RAW264.7 macrophage cell. The data are expressed as mean±SD for four independent experiments. In the figure, “**” and “***” above the bar mean p<0.01 and p<0.001, respectively, indicating statistically significant differences from treatments with LPS alone.

FIG. 7 shows the effect of Pineapplin PL6 on LPS-induced iNOS expression in RAW264.7 macrophage cell. The data are expressed as mean±SD for four independent experiments. “*” above the bar means p<0.05, indicating statistically significant differences from treatments with LPS alone.

FIG. 8 shows the inhibitory effect of Pineapplin PL6 on LPS-induced NFκB expression in RAW264.7 macrophage cell. The data are expressed as mean±SD for four independent experiments. “*” above the bar means p<0.05, indicating statistically significant differences from treatments with LPS alone.

FIG. 9A shows the resulting interaction forces and the bonding types of PGE2 in the binding site of EP4 model; and FIG. 9B shows the resulting interaction forces and the bonding types of Pineapplin PL6 in the binding site of EP4 model, simulated by a Molecular simulation software Discovery Studio.

FIG. 10A shows the Docking pose of PGE2 and FIG. 10B shows the Docking pose of PE6 with the key residues in EP4 molecular. Alkyl interaction, conventional interaction, carbon interaction, negative-negative interaction and charge-charge interaction are marked by a dotted line with the name beside, respectively.

### DETAILED DESCRIPTION OF THE PREFERRED EMBODIMENTS

The present invention provides an anti-inflammatory compound of general formula (I):

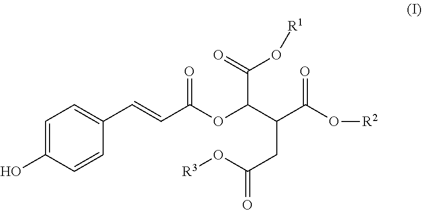

wherein the R¹, R² and R³ are independently selected from a group consisting of H, halo, alkyl, alkenyl, alkynyl, cyclyl, heterocyclyl, alkoxyl, aryl, heteroaryl, alkylaryl and CF₃.

As used herein, the term “halo” means fluorine, chlorine, bromine or iodine.

As used herein, the term “substituted” means that one or more hydrogen atoms on a functional group is substituted by one or more substituents, which may be the same or different. Examples of the substituent include, but are not limited to, halogen, cyano, nitro, hydroxyl, amino, mercapto, alkyl, alkenyl, alkynyl, aryl, heteroaryl, cycloalkyl, heterocyclyl, alkyloxy, aryloxy, alkylsulfonyl, arylsulfonyl, alkylamino, arylamino, dialkylamino, diarylamino, alkylcarbonyl, arylcarbonyl, heteroarylcarbonyl, alkylcarboxy, arylcarbonyl, heteroarylcarboxy, alkyloxycarbonyl, aryloxycarbonyl, heteroaryloxycarbonyl, alkylcarbonyl, alkylaminomethanyl, arylcarboxamide, aminocarboxamide, and the like. Each of the alkyl, alkenyl, aryl, heteroaryl, cycloalkyl and heterocyclic groups may optionally have substituents of halogen, cyano, nitro, hydroxyl, amino, mercapto, alkyl, aryl, heteroaryl, alkyloxy, aryloxy, alkylcarbonyl, arylcarbonyl, alkylcarbonyl, arylcarboxy, alkyloxycarbonyl or aryloxycarbonyl.

As used herein, the term “alkyl” refers to a substituted or unsubstituted linear or branched saturated hydrocarbon group. Preferably, the alkyl group is a substituted or unsubstituted C₁₋₆ alkyl group, including, but not limited to, substituted or unsubstituted methyl, ethyl, n-propyl, isopropyl, n-butyl, secondary butyl, tertiary butyl, n-pentyl, isopentyl, n-hexyl, and the like.

As used herein, the term “alkenyl” or “alkynyl” refers to a substituted or unsubstituted linear or branched unsaturated hydrocarbon group containing at least one double bond or triple bond. Preferably, the alkenyl group is a substituted or unsubstituted C₂₋₆ alkenyl group, including, but not limited to, substituted or unsubstituted vinyl, allyl, butenyl, and pentenyl, 1,4-hexadienyl, and the like. Preferably, the alkynyl group is a substituted or unsubstituted C₂₋₆ alkynyl group, including (but not limited to) substituted or unsubstituted ethynyl, propynyl, butynyl, and the like.

As used herein, the term “cycloalkyl” refers to a partially or fully saturated monocyclic or bicyclic ring system. Preferably, the cycloalkyl group is a substituted or unsubstituted C₄₋₈ cycloalkyl group, including, but not limited to, substituted or unsubstituted cyclobutyl, cyclopentyl, cyclohexyl, and the like.

As used herein, the term “heterocyclyl” refers to a cyclic functional group containing one or more heteroatoms (for example, O, N, or S) as part of the ring system, and the remainder being carbon atoms. Examples of heterocyclic groups include, but are not limited to, substituted or unsubstituted azetidinyl, hexahydropyridinyl, tetrahydropyrrolyl, tetrahydrofuranyl, azepanyl, 1,4-oxazepane, and the like.

As used herein, the term “alkoxy” refers to a group formed by linking a substituted or unsubstituted alkyl group with an oxygen atom. Preferably, the alkoxy group is a substituted or unsubstituted C₁₋₆ alkoxy group, including (but not limited to) substituted or unsubstituted methoxy (—OCH₃), ethoxy, propoxy, butoxy, pentoxy, hexyloxy, and the like.

As used herein, the term “aryl” refers to a cyclic hydrocarbon group having at least one aromatic ring system, which can be monocyclic or bicyclic. Examples of aryl group include, but are not limited to, substituted or unsubstituted phenyl, naphthyl, anthryl, pyrenyl, and the like.

As used herein, the term “Heteroaryl” refers to a cyclic hydrocarbon group having at least one aromatic ring system, which can be a monocyclic, bicyclic or condensed ring system, and the aromatic ring contains at least one heteroatom (for example, 0, N or S) that is part of the ring system, and the remainder being carbon atoms. Examples of heteroaryl groups include, but are not limited to, furyl, pyrrolyl, thienyl, oxazolyl, imidazolyl, pyrazolyl, pyridyl, pyrimidinyl, thiazolyl, furyl, indolyl, and the like.

As used herein, the term “pharmaceutically acceptable” means suitable for contact with human or animal tissues without causing excessive toxicity, irritation, allergic reactions or other complications, within the scope of reasonable medical judgment.

As used herein, the term “pharmaceutically acceptable salt, ester, or hydrate” refers to the salt or ester formed by reacting the acidic group of the compound of general formula (I) with a base or an alcohol, or the hydrate formed by associating a functional group to water through coordination. For example, pharmaceutically acceptable salts include, but are not limited to, alkali metal salts (such as sodium salt, potassium salt), alkaline earth metal salts (such as calcium salt, magnesium salt), ammonium salt, and organic base salts (such as salts formed with cyclohexylamine, N-methyl-D-glucosamine, and the like).

The present invention also provides an anti-inflammatory composition comprising the compound of general formula (I) or its salt, ester or hydrate, and a pharmaceutically acceptable carrier, excipient or diluent. As used herein, the term “pharmaceutically acceptable carriers, excipients or diluents” refer to the pharmaceutically acceptable materials, substrates, such as liquids, solid fillers, stabilizers, dispersants, suspensions, thickener, solvent or encapsulating material, that act to transport the active ingredient of the present invention and make the active ingredient play its function in a subject. The carrier must be compatible with each formulation component in the composition of the invention, including the compound of general formula (I), so that it does not have a negative impact on the subject.

Pharmaceutically acceptable carriers include: sugars, such as lactose, glucose and sucrose; starches, such as corn starch and potato starch; celluloses, such as sodium carboxymethyl cellulose, ethyl cellulose and cellulose acetate; powdered tragacanth; Malt; Gelatin; Talc, and the like. Pharmaceutically acceptable excipients or diluents include: cocoa butter and suppository wax; oils, such as peanut oil, cottonseed oil, safflower oil, sesame oil, olive oil, corn oil, and soybean oil; glycols, such as propylene glycol; polyols, such as glycerin, sorbitol, mannitol and polyethylene glycol; esters, such as ethyl oleate and ethyl laurate; agar; buffers, such as magnesium hydroxide and aluminum hydroxide; surfactants; alginic acid; pyrogen-free water; isotonic saline; Ringer's solution; ethanol; phosphate buffer solution; and other non-toxic pharmaceutically compatible substances.

As used herein, the term “anti-inflammatory” refers to the effects of substances or treatments in inhibiting or reducing the symptoms and occurrence of inflammatory responses “Inflammatory response” refers to the defensive response of living tissues having vascular system to inflammatory factors and local damage, including symptoms of redness, swelling, fever, pain and others Inflammation can be divided into acute inflammation and chronic inflammation. Acute inflammation is the initial response of an organism to harmful stimulations. It causes more plasma and white blood cells, especially granulocytes, to move from the blood to the damaged tissue. Chronic inflammation leads to changes in cell types in the inflamed area, and the tissues destruction and repairing proceed simultaneously. At present, anti-inflammatory effects are investigated by using lipopolysaccharide (LPS) to induce macrophages, and evaluating the inhibitory effects on inflammatory substances production, such as nitric oxide (NO), inducible nitric oxide synthase (iNOS), prostaglandin E2 (PGE2), cyclooxygenase-2 (COX-2), and the expression of NFκB protein.

The other characteristics and advantages of the present invention will be further illustrated and described in the following examples. The examples described herein are intended for illustrations, not for limitations of the invention.

### Example 1. Preparation of Anti-Inflammatory Compound by Partition Extraction from Pineapple Extract

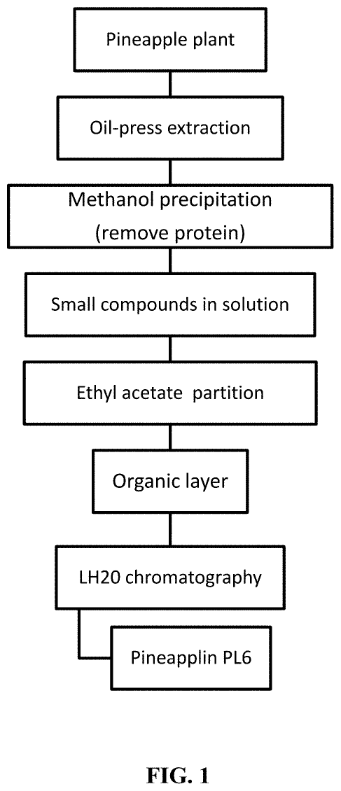

In this example, a single compound PL6 is purified from the water extract of pineapple by partition extraction with dichloromethane and ethyl acetate, and then the isolation on Sephadex LH-20 (30 cm*3 cm id) column of the organic layer obtained from the partition extraction. See FIG. 1 for its flow chart. The detailed preparation method and separation conditions are described below.

After the juice is squeezed from the pineapple plant, the residue is removed by coarse filtration to obtain the pineapple water extract. This solution is then freeze-dried to obtain the pineapple extract powder. Composition analysis of the pineapple extract powder by high performance liquid chromatography (HPLC): feed preparation: re-dissolve the powder with water to a final concentration of 200 mg/ml, followed by filtration with a 0.45 μm syringe filter (13 mm syringe filter with 0.45 μm PP membrane, PALL); analysis conditions: chromatography column, Mightysil RP-18 GP (4.6 mm*250 mm, particle size: 5 μm); injection volume, 20 μl; flow rate, 0.8 ml/min; detection wavelength, 320 nm; 100% acetonitrile (solution A); 1% (v/v) formic acid aqueous solution (solution B); gradient elution conditions: 95-70% B (50 min), 70-50% B (60 min), 50-95% B (70 min).

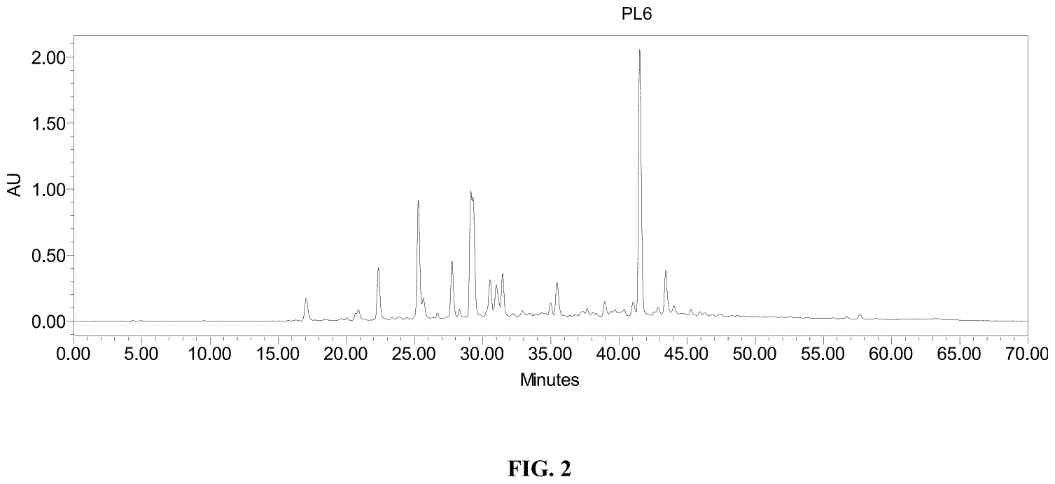

The phenolic compounds in pineapple extract show strong absorption signals at 320 nm, therefore the wavelength of 320 nm is selected for analysis. As shown in FIG. 2, in the pineapple extract, there are major peaks at the residence time of 17, 20.5, 24.5, 28, 34.8, 41, and 42.8 minutes, which are initially named PE1, PE2, PE3, PE4, PE5, PL6 and PE7.

Partition extraction of pineapple compounds includes the following steps: re-dissolve 60 g of the freeze-dried pineapple extract powder in 300 ml of double distilled water, add 600 ml of dichloromethane for partition extraction, and then add 600 ml of ethyl acetate to the water layer for secondary extraction. The organic layer is collected to further concentrate in a vacuum concentrator, and is then stored at 4° C.

Column chromatography of the pineapple extract: The organic layer is further purified by Sephadex LH-20 (30 cm*3 cm id), and extracted with twice the column volume. The extraction gradient is and 20% (v/v) methanol in pure water. The samples are collected by 10 ml per fraction, and the target compound is confirmed by HPLC. The HPLC analysis conditions are: 100% acetonitrile (solution A), water containing 1% (v/v) formic acid (solution B); the elution conditions: 95-70% solution B (50 min), 70-50% solution solution B (60 min), 50-95% solution B (70 min).

Analysis of pineapple extract by liquid chromatography-mass spectrometry (LC-MS): the conditions of liquid chromatography are the same as those described above for HPLC analysis conditions. ESI (Electrospray ionization) negative ion method is used in the mass spectrometer, with ionization temperature of 300° C. and spray voltage of 4.5 kV. The gas flow rates of sheath gas, auxiliary gas and sweep gas are 50, 13 and 3 arbitrary units, respectively. The data-dependent acquisition (DDA) is used for optimal screening conditions, and the signals at 100-1500 m/z in MS' scanning are obtained in a data-dependent manner.

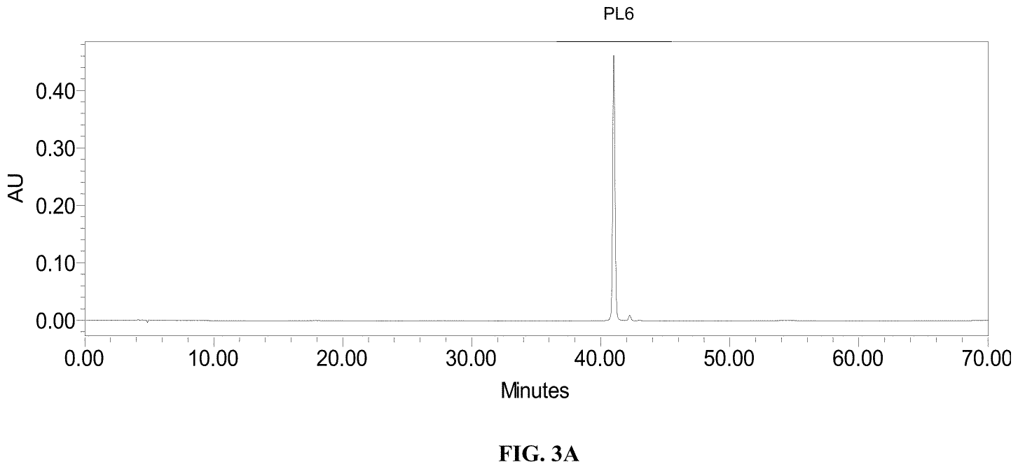

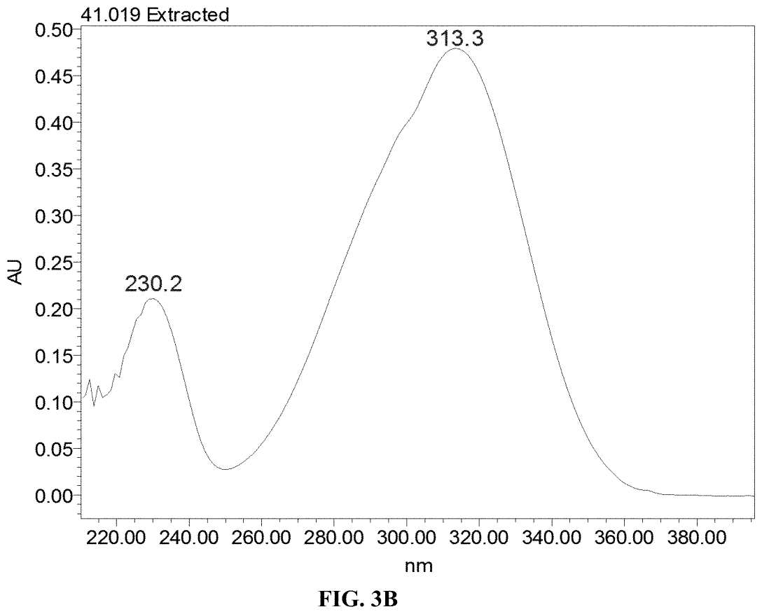

After the partition extraction of pineapple extract with dichloromethane and ethyl acetate, compounds PL6 and PE7 in the organic layer are separated from the rest of the compounds (i.e. PE1-PE5). Further purification of the organic layer by Sephadex LH-20 chromatography will result in a purified active compound of present invention, named Pineapplin PL6, which corresponds to the main signal in the sample analyzed (FIG. 3A). According to the structure predicted from the mass spectrum data, it is inferred that the compound may have anti-inflammatory activity. It is showed that the purified compound has absorption peaks at 313 nm and 230 nm in the further HPLC-PDA chromatogram (FIG. 3B).

Nuclear Magnetic Resonance (NMR) Spectroscopy Analysis of the purified compound from pineapple extract: the Bruker AV-400 MHz NMR spectrometer is used for ¹³C and 2D NMR spectroscopy, and the Jeol JNM-ECA 600 NMR spectrometer is used for ¹H NMR spectroscopy. Tetramethylsilane is used as the internal standard, and the chemical shift is recorded based on 6 values (parts per million, ppm).

The purified Pineapplin PL6 is obtained as a pale yellow powder. According to 1H-NMR spectroscopy, signals of the hydrogen atom on the benzene ring are δ 6.80 (2H, d, J=9.0 Hz) and δ 7.48 (2H, d, J=9.0 Hz), signals of the hydrogen atom on the alkene group are δ 6.39 (1H, d, J=16.2 Hz) and δ 7.67 (1H, d, J=16.2 Hz), and signals of the other hydrogen atom are δ 2.59 (1H, dd, J=17.4, 5.4 Hz), δ 2.81 (1H, dd, J=17.4, 9.0 Hz) and 3.55 (1H, m), indicating there are three carboxyl groups.

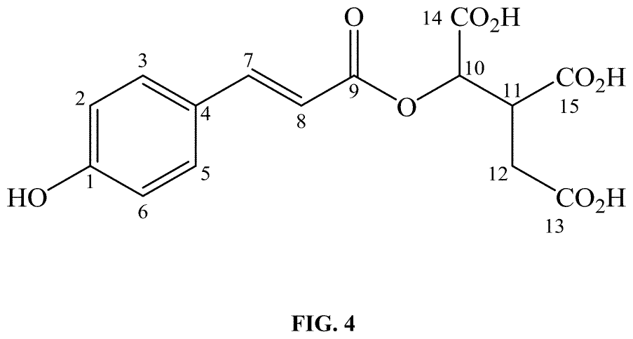

Then, the signals of carbon atom are further confirmed by ¹³C-NMR spectrum. Using Correlation Spectroscopy (COSY), Nuclear Overhauser Effect Spectroscopy (NOESY), and Heteronuclear Multiple Bond Correlation (HMBC), the interaction between hydrogen atoms and the interaction between carbon atoms and hydrogen atoms are confirmed. After confirmation by the data, the purified compound Pineapplin PL6 has chemical nomenclature of 1,2,3-tricarboxylic acid-propyl-3-hydroxyphenol acrylate, and its molecular formula is C₁₅H₁₄O₉, with molecular weight of 338.27, as shown in FIG. 4.

### Example 2. Cytotoxicity Test of Pineapplin PL6

RAW264.7 cells are cultured in RPMI medium containing 10% fetal bovine serum, 0.2% sodium bicarbonate and 1% penicillin/streptomycin, and in a 5% CO₂, 37° C. incubator. The cells are subcultured when a confluency of 70-80% is reached. RAW264.7 cells are inoculated in a 96-well plate at a density of 4×10⁴ cells/well per well. After the cells are adhered for overnight incubation, Pineapplin PL6 at different concentrations (25, 50, 100, 200, 400 and 800 μM) or pineapple extract (3, 6, 12 mg/ml) is added to each well. The blank control group is treated with culture medium without the Pineapplin PL6 or pineapple extract After 24 hours of incubation, Alamar blue is used to test cytotoxicity of the Pineapplin PL6. The medium is removed, washed twice with PBS, and diluted 10 times with Alamar Blue reagent in the medium without FBS. After reaction in dark for 6 hours, the change in absorbance at a wavelength of 570 nm is measured by using an ELISA reader.

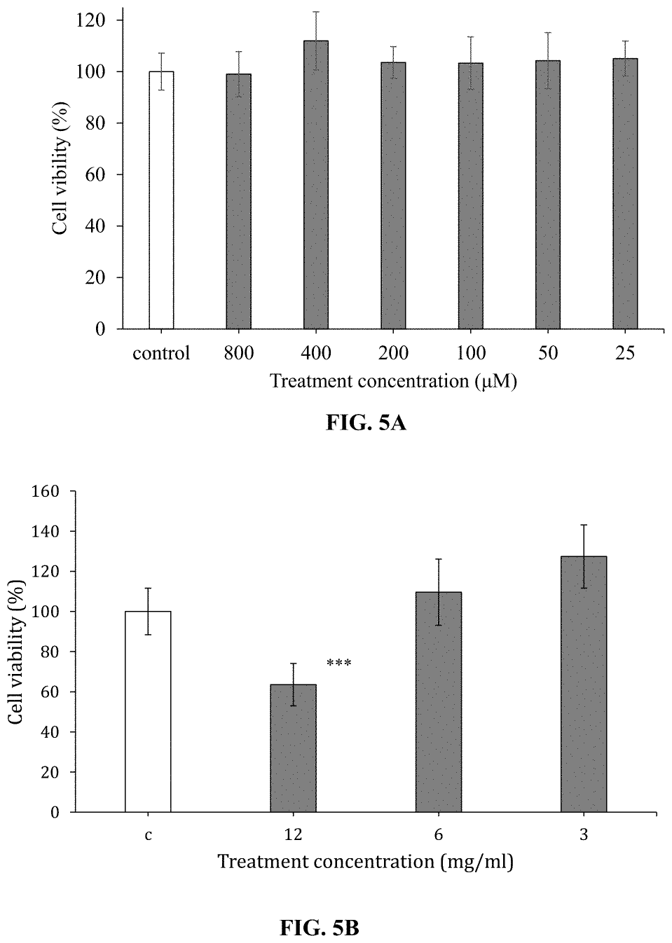

According to the results showed in FIG. 5A, Pineapplin PL6 has no obvious cytotoxicity to RAW264.7 cells at the tested concentrations. As the content of pineapple PL6 in pineapple extract is about 0.1%, the concentrations of pineapple extract were expanded in the cytotoxicity test accordingly. After re-dissolving the pineapple extract in water, the concentrations are increased to 3, 6 and 12 mg/ml, and then Alamar blue is used to perform the cytotoxicity test. It is found that when the concentration of pineapple extract is increased to 12 mg/ml, it showed significant cytotoxicity (as shown in FIG. 5B). In the figure, “***” above the bar means p<0.001, indicating statistically significant differences from treatments with control group.

### Example 3. Pineapplin PL6 Inhibits the Inflammatory Response of Cells Induced by LPS

Effect on LPS-Induced NO Production

RAW264.7 cells are inoculated in a 96-well plate at a density of 4×10⁵ cells/well. After the cells are adhered overnight, 200 ng/ml LPS is added, the blank control group has no LPS added, and incubated at 5% CO₂ and 37° C. for 24 hours. On the second day, different concentrations (50, 100, 200 and 400 μM) of Pineapplin PL6 are added to the wells. The blank control group is treated with LPS-free medium, and the blank control group is treated with PL6-free LPS medium. The cells are then cultured under the condition of 5% CO₂ and 37° C. After 24 hours of incubation, 150 μl of medium for each group is collected.

A solution of 1% p-aminobenzene sulfonic acid dissolved in 5% phosphoric acid is prepared, and then mixed with 0.1% N-(1-naphthyl)ethylenediamine dihydrochloride at a ratio of 1:1 to form the Griess reagent. Solutions of sodium nitrite at concentrations of 1.5625 μM to 100 μM are prepared for making a standard curve. 150 μl of the collected medium is mixed with 50 μl of the Griess reagent, and reacted in a dark environment for 30 minutes. The change in absorbance at a wavelength of 555 nm is measured using an ELISA reader.

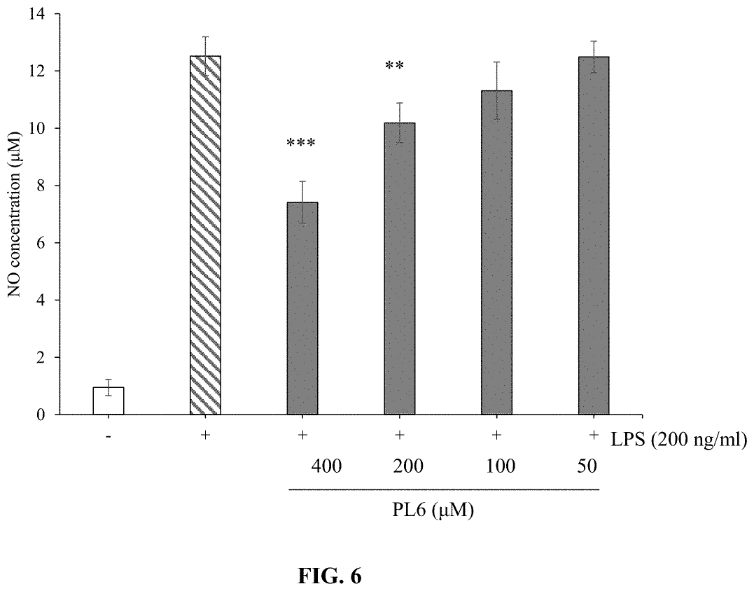

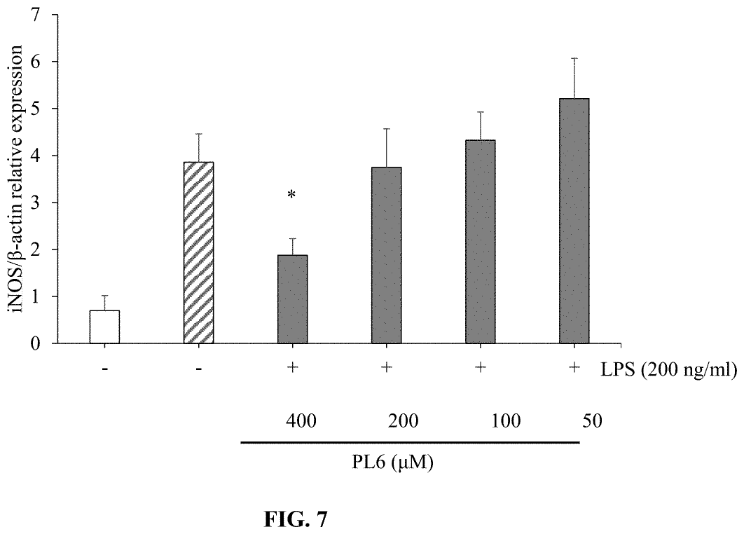

The results show that Pineapplin PL6 significantly reduces the production of nitric oxide induced by LPS in RAW264.7 macrophages at concentrations of 200 μM and 400 μM (FIG. 6), indicating that Pineapplin PL6 exhibits the effects of inhibiting the immune response produced by external stimuli.

Effects on LPS-Induced iNOS Expression

The effect of Pineapplin PL6 on the LPS-induced iNOS expression is tested in RAW264.7 macrophage cells. The iNOS/β-actin ratio is the relative expression level of iNOS and β-actin protein. The higher the expression level of iNOS, the more inflammation response occurred (as an inflammation index). RAW264.7 macrophage cells were pretreated with LPS (200 ng/ml) for 24 hours, then incubated with various concentrations (50, 100, 200 and 400 μM) of PL6 at 5% CO₂ and 37° C. for 24 hours. The (−) control group is treated with LPS-free medium, and the (+) control group is treated with PL6-free LPS medium. The cell culture supernatant of each group is collected, then the protein concentration is calculated, and 30 μg of total protein is taken from each group for protein gel electrophoresis. After the transfer of protein to a solid support membrane is completed, the primary antibody anti-iNOS antibody (diluted at 1:1000) and the secondary antibody anti-rabbit IgG (diluted at 1:5000) are used for Western blot analysis. After washing with PBST, the developing agent (Western Chemiluminescent HRP Substrate) is added. The luminescence fluorescence digital analysis system (ImageQuant LAS 400 mini, GE Healthcare Life Sciences) is used for luminescence color development.

The data shown in FIG. 7 is the relative expression of iNOS protein to β-actin, which is calculated by a software, based on the values obtained from the luminescence color film using scanning optical density method and standardized to β-actin protein. The higher value indicates that higher level of inflammation response occurred. The results of inhibiting iNOS protein expression also show that Pineapplin PL6 does have the effect of suppressing inflammation.

Effects on LPS-Induced NFκB Expression

In this example, effect of Pineapplin PL6 on the LPS-induced NFκB expression is further tested in the RAW264.7 macrophage cell line. The value of p-p65/p65 is the relative expression level of NFκB protein. The higher the expression level of NFκB, the more inflammation response occurred (as an inflammation index). RAW264.7 macrophage cells were pretreated with LPS (200 ng/ml) for 24 hours, then incubated with various concentrations (50, 100, 200 and 400 μM) of PL6 for 24 hours at a condition of 5% CO₂ and 37° C. The (−) control group is treated with LPS-free medium, and the (+) control group is treated with PL6-free LPS medium. The cell culture supernatant of each group is collected, then the protein concentration is calculated, and 30 μg of total protein is taken from each group for protein gel electrophoresis. After the transfer of proteins from the gel to a solid support membrane is completed, the primary antibodies anti-phospho-NFκB p65 and anti-NFκB p65 (diluted at 1:1000), and the secondary antibody anti-rabbit IgG (diluted at 1:5000) are used for Western blot analysis. After washing with PBST, the developing agent (Western Chemiluminescent HRP Substrate) is added, and the luminescence fluorescence digital analysis system (ImageQuant LAS 400 mini, GE Healthcare Life Sciences) is used for luminescence color development.

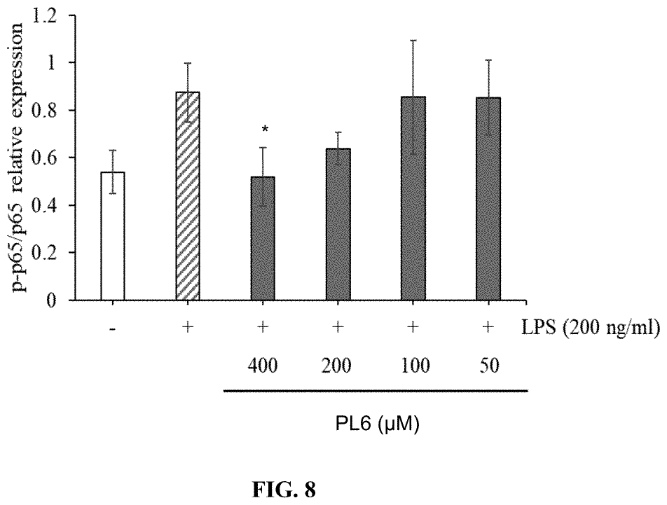

The data shown in FIG. 8 is the relative expression of NFκB protein p-p65/p65, which is calculated by a software, based on the values obtained from the luminescence color film using scanning optical density method and standardized to β-actin protein. The higher value indicates that the higher level of inflammation response occurred. The results of inhibiting NFκB protein expression also show that Pineapplin PL6 has the effect of suppressing inflammation.

### Example 3. Simulation Calculation of Molecular Docking of Pineapplin PL6 and Prostaglandin E2 Receptor EP4

Since Pineapplin PL6 described in Example 1 is structurally similar to PGE2, and PGE2 analogues have previously been reported to have the potential of anti-inflammatory activity, we use the prostaglandin E2 (PGE 2) receptor EP4 as the active center target of PGE2 to compare the binding abilities of PL6 and PGE2 to EP4 and the binding energy with the receptor through the calculation of molecular simulation software GEMDOCK.

The calculated results listed in Table 1 below show that the chemical energy required for PGE2 is −104.7 kJmol⁻¹ (including van der Waals force −83.3 kJmol⁻¹, hydrogen bond −20.9 kJmol⁻¹, electrostatic force −0.6 kJmol⁻¹); and the chemical energy required for PL6 is −108.1 kJmol⁻¹ (including van der Waals force −82.5 kJmol⁻¹, hydrogen bond −21.9 kJmol⁻¹, electrostatic force −3.0 kJmol⁻¹).

TABLE 1
Chemical energy calculated by GEMDOCK for the interaction
between the binding pocket of the prostaglandin E2 receptor
EP4 and ligands PL6 and PGE2.

| | Total Energy | VDW | H Bond | Elec |
|---|---|---|---|---|
| Ligand | (kJ mol⁻¹) | (kJ mol⁻¹) | (kJ mol⁻¹) | (kJ mol⁻¹) |
| PGE2 | −104.7 | −83.3 | −20.9 | −0.6 |
| PL6 | −108.1 | −82.5 | −21.9 | −3.0 |

VDW: Van der Waals force
Elec: electro statistic energy

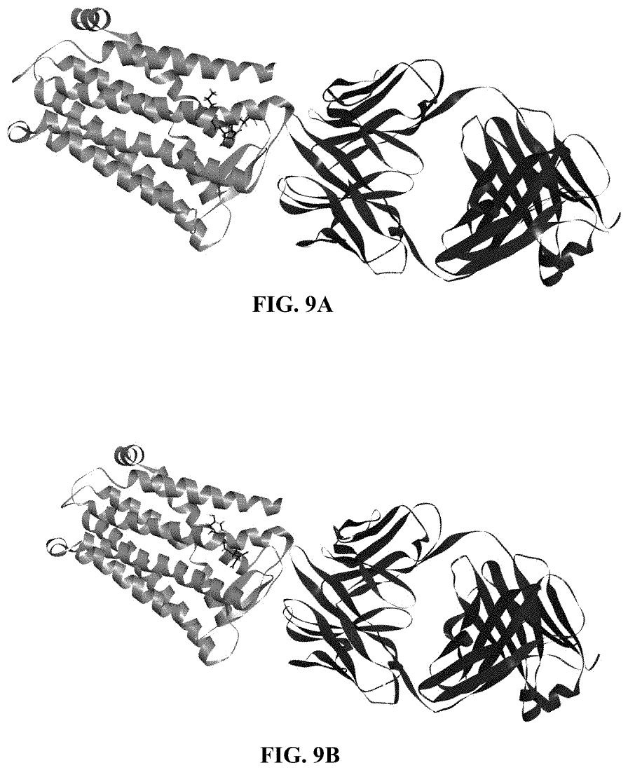

When using the molecular simulation software Discovery Studio to compare the bonding force, as well as the bonding type and strength, of PGE2 and PL6 generated between these two compounds and the EP4 binding site, it is shown that, according to FIG. 9A and FIG. 9B, both PGE2 (shown in FIG. 9A) and PL6 (shown in FIG. 9B) can enter the binding site of EP4.

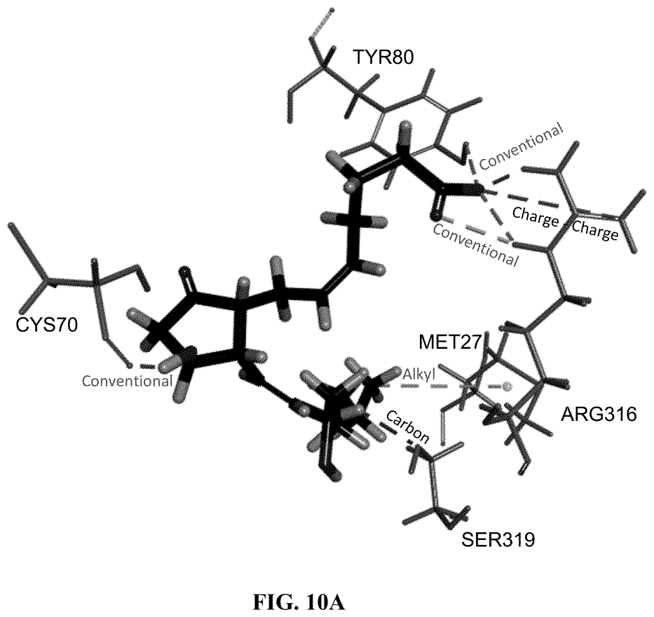

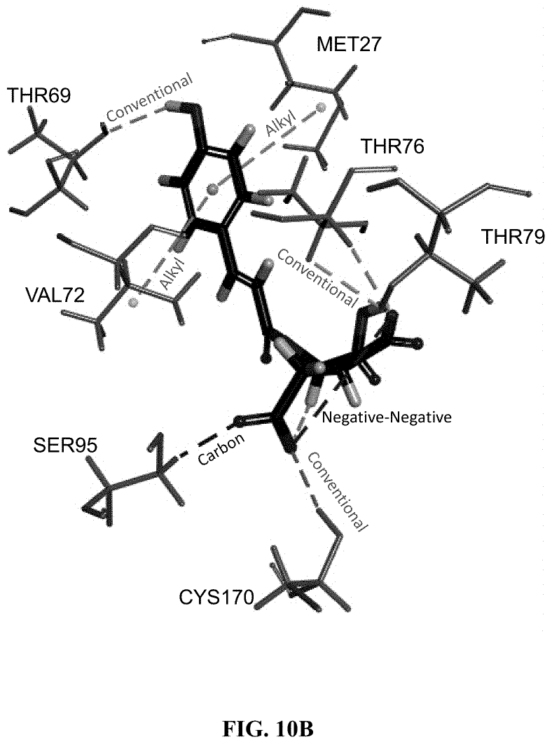

There are four main intermolecular forces between PGE2 and EP4, which are alkyl interaction, hydrogen bond (conventional bond), non-classical hydrogen bond, and charge-charge interaction. As shown in the molecular docking model in FIG. 10A, there is an alkyl group force between PGE2 and EP4, and the alkyl group force is formed between the carbon chain of PGE2 and the MET27 of EP4 molecule. Three forces, including two hydrogen bonds and one charge-charge interaction, form on the ARG316 of EP4 and the carboxylic acid group of PGE2. In addition, the carboxylic acid group of PGE2 also forms a hydrogen bond with the TYR80 of EP4. A hydrogen bond is formed between the hydroxyl group on the PGE2 ring and the CYS170 of EP4; and the carbon chain at the other end of PGE2 will form a non-classical hydrogen bond with the SER319 of EP4.

And between PL6 and EP4, as shown in the molecular docking model in FIG. 10B, there are four main intermolecular forces: π-alkyl interaction, hydrogen bonding (conventional bond), non-classical hydrogen bond, and negative-negative interaction. The π-alkyl interactions are formed between the benzene ring of PL6 and the two amino acids MET27 and VAL72 of EP4. The hydroxyl group on the benzene ring of PL6 forms a hydrogen bond with the THR69 of EP4. The carboxyl groups on the carbon chain of PL6 form four hydrogen bonds with the THR76, THR79 and CYS170, and one non-classical hydrogen bond with the SER95 of EP4.

Although a limited number of embodiments are described to illustrate the practice of the present invention, those skilled in the art may still make modifications or changes according to the description. Therefore, the scope of the present invention should only be limited by the claims of the patent, and not limited to the above examples.

What is claimed is:

1. An anti-inflammatory compound of general formula (I), 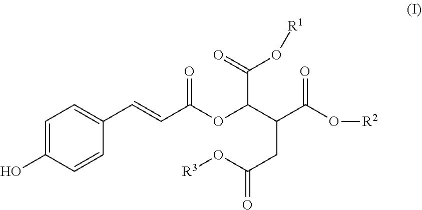
wherein the R¹, R² and R³ are same or different, and independently selected from a group consisting of H, F, Cl, Br, I, alkyl, alkenyl, alkynyl, cyclyl, heterocyclyl, alkoxyl, aryl, heteroaryl, alkylaryl and CF₃.

2. The compound of general formula (I) of claim 1, wherein at least one of the R¹, R² and R³ is H.

3. The compound of general formula (I) of claim 1, wherein all of the R¹, R² and R³ are H.

4. The compound of general formula (I) of claim 1, wherein at least one of the R¹, R² and R³ is a substituted or unsubstituted C₁₋₁₀ alkyl.

5. The compound of general formula (I) of claim 1, wherein all of the R¹, R² and R³ are substituted or unsubstituted C₁₋₁₀ alkyl.

6. The compound of general formula (I) of claim 4, wherein the substituted or unsubstituted C₁₋₁₀ alkyl is a substituted or unsubstituted C₁₋₆ alkyl.

7. The compound of general formula (I) of claim 1, wherein the alkyl is selected from a group consisting of substituted or unsubstituted methyl, ethyl, propyl, isopropyl and butyl.

8. The compound of general formula (I) of claim 1, wherein the alkoxyl is selected from a group consisting of substituted or unsubstituted methoxy, ethoxy, propoxy, isopropoxy, butoxy, pentoxy and hexoxy.

9. The compound of general formula (I), or its pharmaceutically acceptable salt, ester, hydrate of claim 1, wherein the alkenyl is selected from a group consisting of substituted or unsubstituted vinyl, allyl, butenyl and pentenyl.

10. A method for preparing the compound of general formula (I) of claim 1, comprising steps of:
performing partition extraction of a water extract of a fruit with dichloromethane and ethyl acetate to obtain an organic layer and an aqueous layer;
applying the organic layer to a gel filtration chromatography column to separate compounds in the organic layer; and
performing eluting on the organic compound to obtain the compound of general formula (I).

11. The method of claim 10, wherein the fruit is a pineapple.

12. The method of claim 10, wherein the water extract of a fruit is a pineapple water extract obtained by squeezing the juice from the pineapple plant, and removing the residue by coarse filtration.

13. An anti-inflammatory composition, comprising the compound of general formula (I) of claim 1, and a pharmaceutically acceptable carrier, excipient or diluent.

14. The anti-inflammatory composition of claim 13, wherein the compound of general formula (I) has a binding ability to a prostaglandin E4 receptor (EP₄).

15. The anti-inflammatory composition of claim 13, wherein the compound of general formula (I) is used to suppress an inflammation response.
